(19) United States
(12) Patent Application Publication
(10) Pub. No.: US 2018/0049738 A1
(43) Pub. Date: Feb. 22, 2018

(54) ARTICULATING MEDICAL DEVICE

(71) Applicant: Artack Medical (2013) Ltd., Maccabim-Reut (IL)

(72) Inventors: Raphael F. MELOUL, Shilo (IL); Mordehai SHOLEV, Moshav Amikam (IL); Shahar PELED, Kibbutz Beit HaShita (IL)

(21) Appl. No.: 15/556,631

(22) PCT Filed: Mar. 23, 2016
(86) PCT No.: PCT/IL16/50309

§ 371 Date: Sep. 8, 2017

Related U.S. Application Data
(60) Provisional application No. 62/141,316, filed on Apr. 1, 2015.

(51) Int. Cl. A61B 17/068

(52) U.S. Cl. CPC A61B 17/068; A61B 2017/00734

(57) ABSTRACT

A medical device and method of using same are provided. The medical device includes a handle detachably connected to a shaft having a proximal portion attached to a distal portion through an articulation region. The device further includes an articulation mechanism controllable from the handle. The articulation mechanism includes a first gear disposed in the proximal portion and a second gear disposed in the distal portion. A drive mechanism for deploying an implant from a distal end of the distal portion includes an elongated member having a flexible region traversing the articulation region with the first gear disposed around the elongated member.

### FIELD AND BACKGROUND OF THE INVENTION

The present invention relates to a device for intrabody use and, more particularly, to an articulating device suitable for mechanically securing implants, such as hernia meshes to intrabody tissue as well as an articulating shaft for use with a medical device.

Suturing is a mainstay of surgical repair, however, manipulation of a suture needle as well as access to the suturing location can be difficult in minimally invasive surgery due to the limited anatomical space around the target tissues.

Due to these limitations of suturing, devices developed to deliver staples, fasteners (e.g. tacks), anchors and tissue adhesives have gained wide spread acceptance in minimally invasive surgery. Such devices enable rapid and accurate ligation of tissue and/or fixation of implants to tissue under the anatomical space constraints imposed by minimally invasive surgery.

One minimally invasive surgical approach that utilizes such a device is hernia repair.

A hernia is a protrusion of abdominal content (preperitoneal fat, omentum or abdominal organs) through an abdominal wall defect.

Currently, the most frequently used minimally invasive technique involves laparoscopic fixation with transabdominal devices that deliver helical coils (tacks) with a maximal tissue penetration depth of several millimeters.

Fixation with tacks is fast and strong and can be rapidly achieved, however, due to anatomical constraints, it can be difficult or impossible to correctly align the tack-delivery head of rigid tackers perpendicular to the mesh-tissue interface and thus the resultant fixation can be less than optimal.

Tacker devices with articulating tack delivery heads were developed to traverse this limitation of rigid devices and provide correct positioning of the tacker delivery head and optimal tack fixation.

Such devices are described in the patent literature (see, for example, US20130119108; US20120271285 and are commercially available (e.g. Covidien ReliaTack™).

Although such devices can be used to select a tack delivery angle (with respect to the mesh-tissue interface), selection can be limited to preset angles which can be suboptimal under some conditions. In addition, the small diameter of the shaft required for minimally invasive delivery and the relatively complex construction of the articulation joint can limit the amount of force applied to the device during angled delivery of the tack.

There it would be highly advantageous to have a tissue ligation/fixation device devoid of the above limitations.

### SUMMARY OF THE INVENTION

According to one aspect of the present invention there is provided a medical device comprising: (a) a handle detachably connected to a shaft having a proximal portion attached to a distal portion through an articulation region; (b) an articulation mechanism controllable from the handle and being for controlling an articulation angle of the distal portion, the articulation mechanism including a first gear disposed in the proximal portion and a second gear disposed in the distal portion; and (c) a drive mechanism operable from the handle and being for deploying an implant from a distal end of the distal portion, the drive mechanism including an elongated member having a flexible region traversing the articulation region, wherein the first gear is disposed around the elongated member.

According to further features in preferred embodiments of the invention described below, the flexible region of the elongated member traversing the articulation region is configured for accommodating a change in angle of the articulation region.

According to still further features in the described preferred embodiments the flexible region is capable of elastically elongating when the distal portion is angled with respect to the proximal portion.

According to still further features in the described preferred embodiments the flexible region forms an arc when the distal portion is co-linear with the proximal portion.

According to still further features in the described preferred embodiments the handle includes a motor for actuating the drive mechanism.

According to still further features in the described preferred embodiments the implant is a tissue anchor.

According to still further features in the described preferred embodiments the distal portion of the shaft is detachable from the proximal portion.

According to still further features in the described preferred embodiments the drive mechanism further includes an implant driver disposed in the distal portion of the shaft.

According to still further features in the described preferred embodiments a distal end of the elongated member engages the implant driver.

According to still further features in the described preferred embodiments the implant driver is rotatable via the elongated member.

According to still further features in the described preferred embodiments rotation of the implant driver delivers the implant from the distal end of the distal portion.

According to still further features in the described preferred embodiments the distal portion of the shaft includes a plurality of implants.

According to still further features in the described preferred embodiments the drive mechanism cannot be activatable during activation of the articulation mechanism.

According to still further features in the described preferred embodiments the drive mechanism is controllable from the handle via a trigger.

According to still further features in the described preferred embodiments activation of the trigger deploys a single implant from the distal end of the distal portion.

According to still further features in the described preferred embodiments the drive mechanism is only deployable when the distal portion of the shaft is correctly attached to the proximal portion.

According to still further features in the described preferred embodiments the articulation mechanism is controllable from the handle via a roller interface.

According to still further features in the described preferred embodiments a position of the roller interface indicates an angle of the distal portion with respect to the proximal portion.

According to another aspect of the present invention there is provided a medical device shaft attachable to a handle, the shaft comprising a proximal portion attached to a distal portion through an articulation region having an articulation control mechanism controllable from a proximal portion of the shaft, the articulation mechanism being for controlling an articulation angle of the distal portion of the shaft.

According to still further features in the described preferred embodiments the articulation mechanism includes a first gear disposed in the proximal portion and a second gear disposed in the distal portion.

According to still further features in the described preferred embodiments the articulation mechanism includes a rod positioned in the proximal portion and being hingedly connected to the distal portion through a lever traversing the articulation region.

According to still further features in the described preferred embodiments the articulation control mechanism is manually activatable to set an angle of articulation of the distal portion with respect to the proximal portion.

According to still further features in the described preferred embodiments manually activating the articulation control mechanism actuates a switch for disabling functions of a handle attachable to the proximal portion of the shaft.

According to still further features in the described preferred embodiments the medical device shaft further comprising a drive mechanism disposed within the shaft, the drive including an elongated member having a flexible region traversing the articulation region, wherein the first gear is disposed around the elongated member.

The present invention successfully addresses the shortcomings of the presently known configurations by providing an articulating tissue fastener device that can be used in minimally invasive procedures for repair of tissue such as abdominal tissue.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is an isometric view of one embodiment of the present device.

FIG. 2 illustrates one embodiment of a handle of the present device.

FIGS. 3a-c illustrate the internal components of the handle of FIG. 2.

FIG. 4a-b illustrate one embodiments of a shaft of the present device in side (FIG. 4a) and cross sectional (FIG. 4b) views.

FIGS. 4c-d are magnified views of the distal portion (FIG. 4c) and handle engaging portion (FIG. 4d) of the shaft shown in FIG. 4b.

FIGS. 5a-d illustrate the articulating region (FIGS. 5a, 5c and 5d) and handle-coupling portion (FIG. 5b) of the shaft of the present device.

FIGS. 6a-b illustrate in greater detail the fastener-carrying cartridge of the distal portion of the shaft shown in FIG. 4c.

FIGS. 7a-d illustrate embodiments of a tissue fastener that can be delivered by the present device.

FIGS. 8a-c illustrates an embodiment of a shaft articulation mechanism deployable via a slider button. FIG. 8b is a magnified view of the region circled in FIG. 8a. FIG. 8c is a closed up view of the articulating region of this embodiment of the present invention.

FIG. 9 illustrates a prototype device constructed in accordance with the teachings of the present invention.

FIGS. 10-11 illustrate tack delivery through a tissue model using the device of FIG. 9 (FIG. 10) and the delivered tack (FIG. 11).

FIGS. 12a-b illustrate an articulating shaft having a shaft-positioned articulation control mechanism (FIG. 12a) and the internal components of the articulation control mechanism (FIG. 12b).

FIG. 13 is an image of a prototype articulating shaft having shaft-positioned articulation control mechanism.

### DESCRIPTION OF THE PREFERRED EMBODIMENTS

The present invention is of a tissue ligation/fixation device which can be used to fixate an implant to a tissue. Specifically, the present invention can be used to deliver a tissue fastener to a body tissue at a variety of angles using a minimally invasive approach.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Devices for fixating implants such as meshes to body tissues using minimally invasive approaches are well known in the art. Such devices can include a rigid or articulating delivery shaft.

In a previously filed application, the present inventors described one such articulating device which includes a drive mechanism for delivering tissue fasteners and an articulation joint having a laterally displaced articulation arm.

While experimenting with several prototypes of an articulation-capable tissue fastener, the present inventors realized that the diameter constraints imposed on the device shaft by the delivery port (5.5 mm or less) and the complexity of the articulation region that supports articulation and enables passage of the fastener drive shaft can result in unwanted deflection of the articulation joint and drive shaft under loads applied during angulation of the delivery head.

In order to minimize the effects of such loads, the present inventors devised an articulation joint and fastener drive shaft arrangement that enable delivery head deflection angles of as much as 95 degrees without compromising the functionality of the articulation joint or drive shaft running therethrough during angulation and forcible loading of the delivery head.

Thus, according to one aspect of the present invention there is provided a medical device which is capable of approximating, ligating and fixating tissues and/or implants such as meshes and the like and can be used in both open and minimally invasive surgeries. The present device can be used in hernia mesh repair, both Inguinal and Ventral, Laparoscopic and open approaches. It can also be used for repairing pelvic or rectal prolapse.

The medical device includes a handle and a shaft having a proximal portion attached to a distal portion through an articulation region. The handle can be permanently attached to the shaft or removably attached thereto. The latter case enables use of several handle types with one shaft and/or reuse of the handle or use of one handle with several shafts.

The medical device further includes an articulation mechanism that is operable from the handle. The articulation mechanism is operable to select an articulation angle of the distal portion of the shaft. As is further described hereinunder, one embodiment of the articulation mechanism includes a first gear a second gear disposed in the articulation region and a third gear disposed on the articulation axis. The gears are engageable to transfer a rotation motion of the first gear in one plane into a respective rotation motion of the second gear and third gear in another plane. Preferably, the first gear rotates around an axis which is substantially perpendicular to an axis of the second and third gears.

The medical device further includes a drive mechanism that is operable from the handle. The drive mechanism is operable to deploy a fastener from a distal end of the distal portion. As used herein, the term fastener relates to any element capable of attaching to a tissue and/or implant. Examples include tacks, staples, anchors, screws and the like.

The drive mechanism includes an elongated member running the length of the shaft from the handle to the distal portion traversing the articulation region. The elongated member runs through the first gear and is in a co-axial arrangement therewith.

The articulation mechanism includes a hollow tube disposed (coaxially) within the proximal portion of the shaft with the first gear being disposed at the distal end of the tube. The gear teeth of the first gear are arranged around the tube or form an end thereof and are designed to selectively engage perpendicularly oriented teeth of the second gear disposed in the distal portion. The handle includes a roller-type interface (e.g. dial) that can be actuated to rotate the tube through a set of drive gears. The tube can be rotated in clockwise or counterclockwise directions (by rolling the dial forwards or backwards) one or more full rotations. The number of rotations required to achieve maximum articulation depends on the gear ratio provided between the first and second gears.

The roller interface can be used to set articulation at any angle between 0-95 degrees (between the proximal and distal portions) e.g. 10, 20, 40, 60, 80, 90 degrees.

The drive mechanism includes a motor, a battery pack and associated electronics and interface elements for controlling and driving the elongated member which in turn drives a fastener delivery mechanism disposed in the distal portion of the shaft.

The interface for the drive mechanism (e.g. trigger) allows a user to deliver a single fastener from the distal end of the shaft with a single push of the button. Delivery is actuated by the motor which rotates the elongated member a predetermined rotation angle or a preselected number of rotations for every push of the button. Rotation of the elongated member rotates the fastener delivery mechanism which in turn rotates and delivers a fastener.

The distal portion of the shaft which includes the fastener delivery mechanism also includes a fastener cartridge holding two or more (preferably 3, 4, 5, 6, 7, 10 or more) fasteners arranged along a length of the distal portion. The fasteners can be coupled to one another such that delivery of one fastener advances all the fasteners in the cartridge and ‘cocks’ the cartridge for subsequent delivery.

Since the distal portion of the shaft also functions as a fastener cartridge, it is preferably detachable from the proximal portion near (distal to) the articulation region. In order to enable such detachment and subsequent attachment of a second distal portion, the elongated member is attached to the fastener delivery mechanism through a detachable coupling such as a bayonet and an Allen pin to hex socket coupling. The distal portion of the shaft is attached to the proximal portion through a one sided or two sided joint which aligns the first and second gears of the articulation mechanism. The joint can be forced apart to disengage the gears and elongated member and detach the distal portion from the proximal portion.

As is mentioned hereinabove, the present inventors designed the articulation region of the device in order to maximize integrity and functionality under the most strenuous delivery conditions.

The positioning of the articulation gears and specifically the co-axial arrangement of the first gear with respect to the elongated member ensures that the first gear and elongated member cooperate to stabilize the articulation region and specifically the elongated member when rotated (by the motor) under loads applied to the device delivery head when the distal portion is angled with respect to the proximal portion.

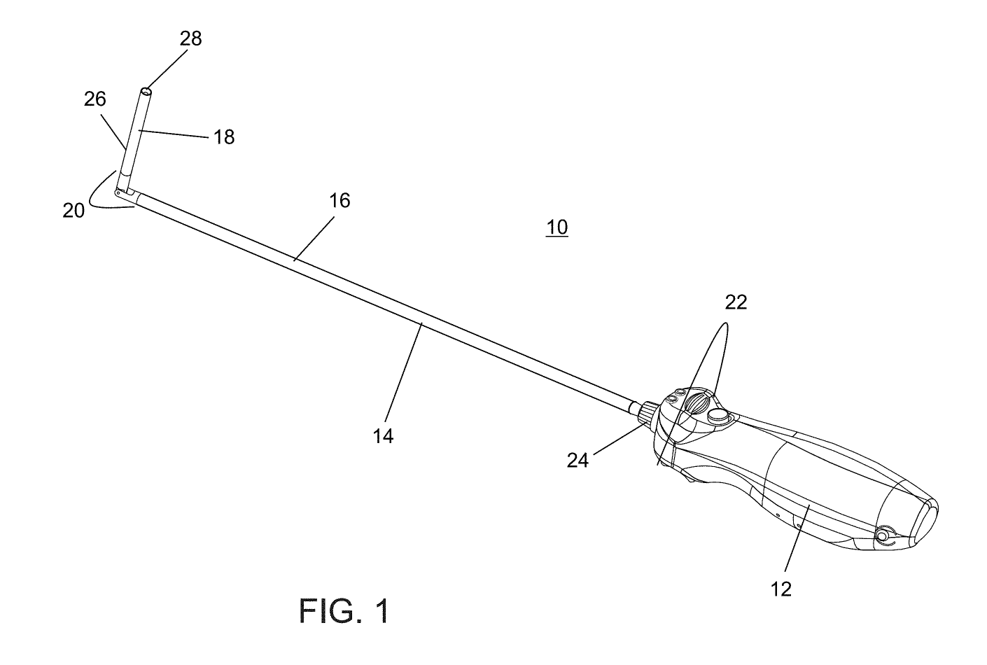

Referring now to the drawings, FIG. 1 illustrates an embodiment of the present device which is referred to hereinunder as device 10.

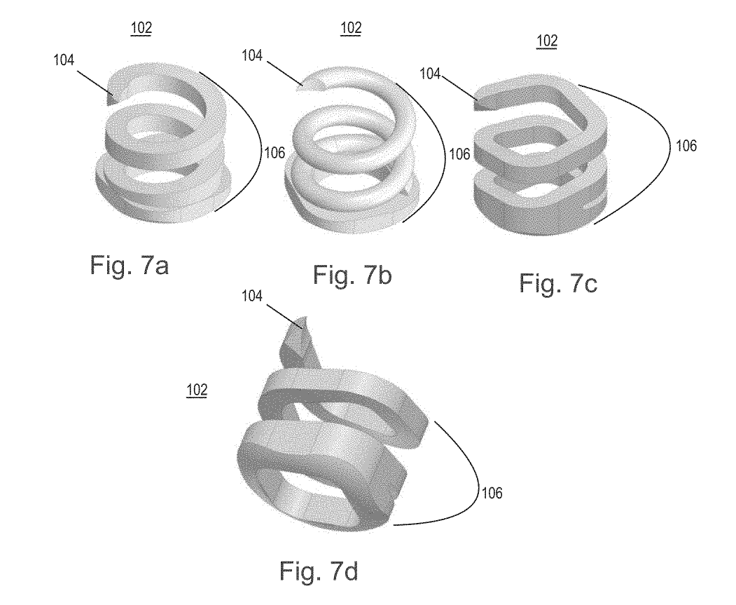

Device 10 is configured for delivering a tack-type tissue fastener (e.g. FIGS. 7a-d) suitable for attaching a surgical mesh such as a hernia mesh to tissue.

Device 10 includes a handle 12 and a shaft 14 having a proximal portion 16 attached to a distal portion 18 through an articulation region 20. Handle 12 can be permanently attached to shaft 14 (e.g. glued) or it can be attached thereto through a releasable coupling.

Handle 12 can be fabricated from a polymer such as Polycarbonate, ABS, Polyurethane using Injection molding, casting machining or 3D printing approaches. Preferably two halves forming the handle shell are fabricated using injection molding and the two halves are glued or mechanically adjoined around the internal components (further described hereinunder). Typical dimensions for handle 12 are 145-200 mm length, 35-55 mm height and 25-50 mm width.

Handle 12 is ergonomically shaped and is operated by wrapping two to four fingers around the handle body with the thumb over the articulation controls of interface 22 and forefinger at the fastener actuation button (trigger) of interface 22.

Shaft 14 can be fabricated from a variety of medical grade stainless steel using machining approaches. Typical dimensions for shaft 14 are 200-300 mm length and 5-10 mm outer diameter. A lumen extends the length of shaft 12 and is 3-6 mm in diameter.

Proximal portion 16 of shaft 14 is connectable to handle 12 via a handle coupling mechanism 24. Proximal portion 16 is typically 200-300 mm in length. Distal portion 18 is connected to proximal portion 16 distally to an articulation region 20.

Distal portion 18 includes a tissue fastener cartridge 26 and mechanism for delivering one or more tissue fasteners through distal opening 28. Distal portion 18 is typically 50-70 mm in length.

Handle 12 controls both articulation of distal portion 18 and delivery of tissue fasteners from cartridge 26.

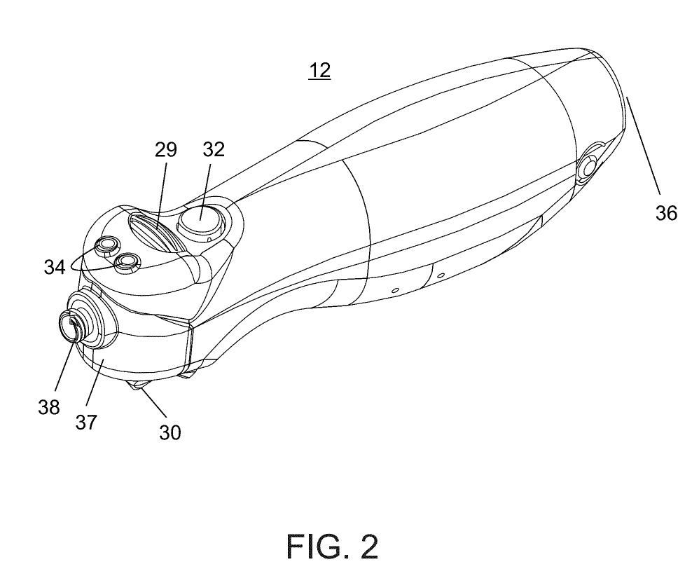

FIG. 2 illustrates handle 12 in greater detail showing interface 22 having a roller-type button 29 operable via a thumb and being for articulating distal portion 18 and a trigger-type button 30 operable via a forefinger and being for actuating release of a tissue fastener from opening 28.

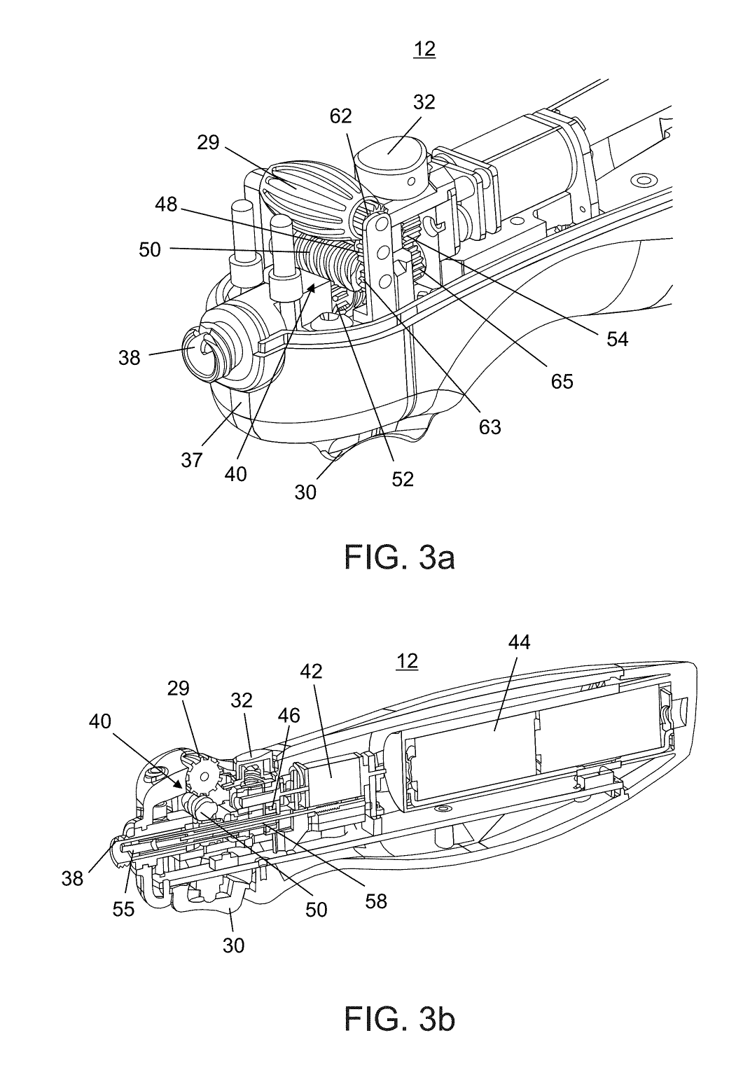

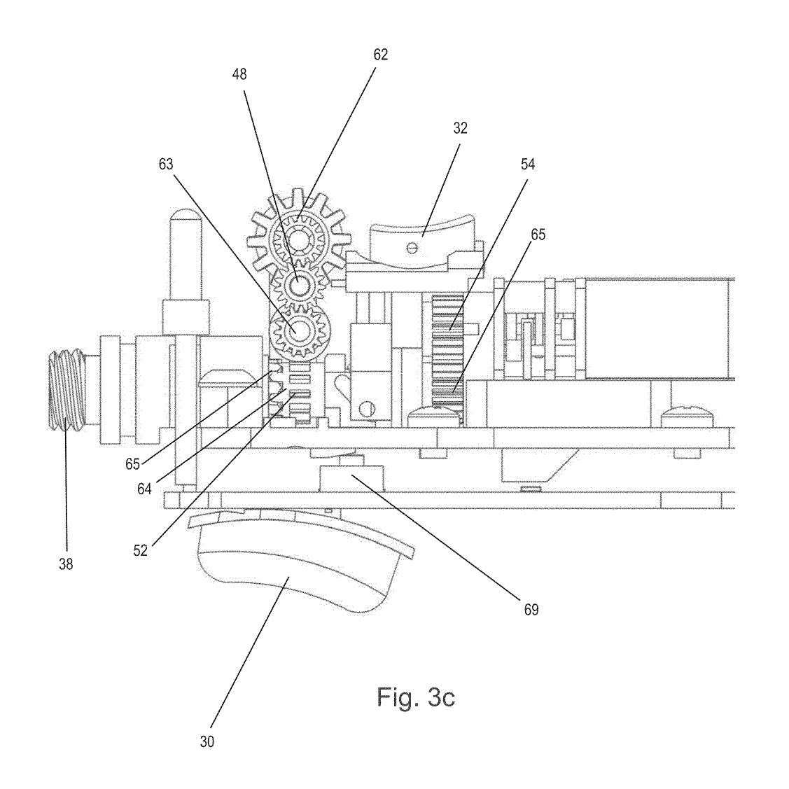

Interface 22 further includes a neutral activation button 32 for engaging/disengaging the articulation gear. When neutral activation button 32 is disengaged, the distal portion of the shaft can articulate freely (simply by pushing the handle against the shaft) and the fastener delivery button is deactivated (via switch 69, FIG. 3c) to prevent delivery of a fastener while the distal portion is articulated. Once an articulation angle is selected by the operator, engaging neutral activation button 32 locks articulation and allows delivery of a fastener from the distal end (as is indicated by a pair of LED lights on the handle).

Handle 12 further includes a port 36 (e.g. USB) for programming a microcontroller of the fastener delivery mechanism in handle 12. Port 36 can be positioned at the proximal end of handle 12 (as is shown in FIG. 2), or on a side face of handle 12.

Distal end 37 of handle 12 includes a coupling mechanism 38 for attaching shaft 12 as well as internal shaft components for transferring actions from roller type button 29 to articulation region 20 and from trigger-type button 30 to cartridge 20. The internal shaft components are further described hereinbelow.

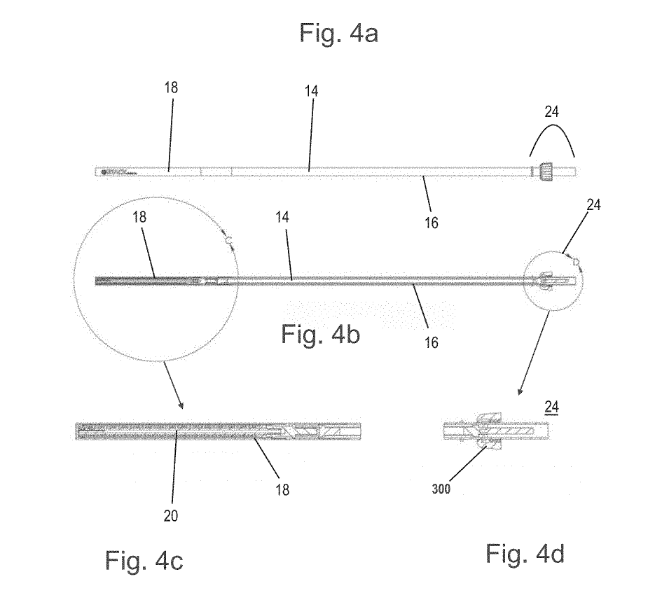

Coupling mechanism 38 includes an outer lug 33 (FIG. 4d) which can be threaded over handle coupling mechanism 24. Coupling mechanism 38 also includes a U-shaped connecting element 55 (FIG. 3b) which interconnects with U-shaped element of shaft 14.

FIGS. 3a-c illustrate the internal components of handle 12, showing roller-type button 29 and associated handle articulation mechanism 40 (FIG. 3a, c) and motor 42, battery 44 and associated handle fastener mechanism 46 (FIG. 3b) for actuating U-shaped connecting element 55 and articulation in shaft 14 attached thereto.

Handle articulation mechanism 40 includes a transfer gear 48 for transferring rolling action of button 29 to a worm gear 50. Worm gear 50 engages a drive gear 52 which is arranged around an articulation drive tube 55 running the length of a lumen of proximal portion 16 of shaft 14. Neutral button 32 when fully depressed engages gear 52 and enables the transfer of torque to articulation connector 55 and when fully released disengages gear 52 providing free or roller button 29 -activated articulation.

Articulation drive tube 55 is a hollow, preferably metal alloy (e.g. stainless steel or titanium) tube having a length of 35-40 mm an outer diameter (OD) of 3.0-4.0 and an inner diameter (ID) of 2.2-2.5 mm.

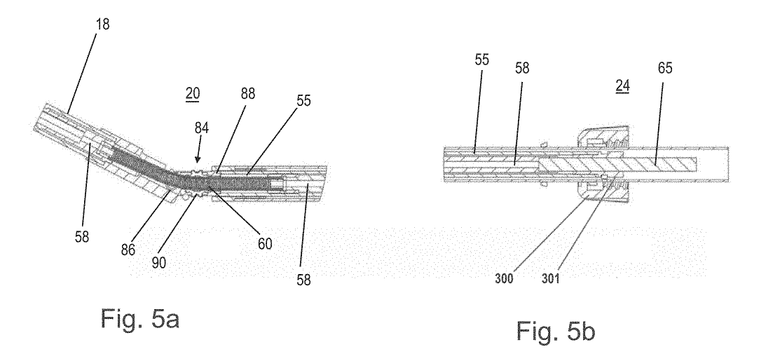

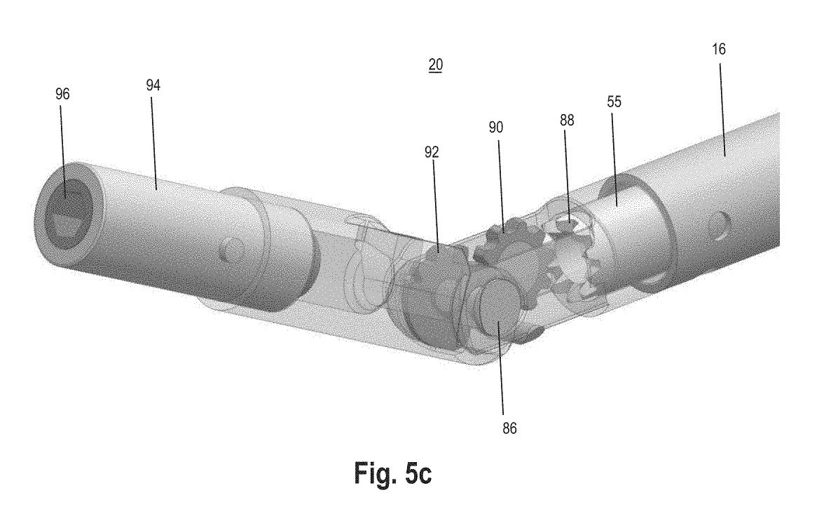

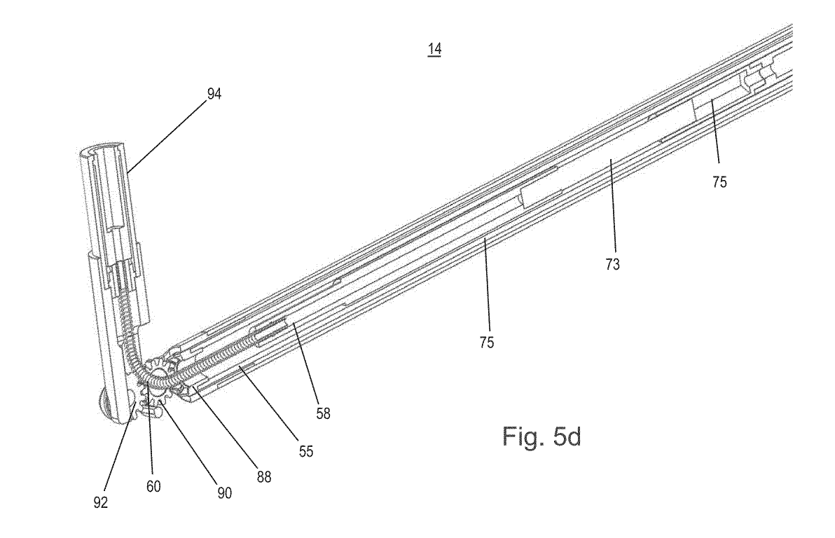

Referring to FIGS. 3a-c, button 29 and articulation mechanism 40 function as follows, thumbing button 29 (forwards or backwards) rotates gear 62 which is attached to thumbing button 29. Gear 62 rotates gear 48 which in turn rotates gear 63. Gear 63 is attached to worm gear 50 which in turn meshes with gear 52. Rotation of gear 52 rotates shaft 64 which is meshed to shaft 65 (FIG. 3c) which is attached to shaft 55. Rotation of shaft 55 rotates crown gear 88 (also referred to herein as first gear) of articulation region 20 (FIGS. 5a, c). Crown gear 88 is meshed to spur gear 90 (also referred to herein as second gear) and causes spur gear 90 to rotate. Spur gear 90 rotates spur gear 86 (also referred to herein as third gear) to thereby articulate distal portion 26 to a desired angle.

Handle fastener mechanism includes a spur gear 54 rigidly attached to shaft of motor 42. Spur gear 46 transfers rotation of motor 42 to an elongated member 58 running the length of a lumen of shaft 12. As is shown in FIGS. 5a and 5d, elongated member 58 includes a flexible portion 60 which traverses articulation region 20. Elongated member 58 is preferably a solid rod or tube fabricated from a metal alloy (e.g. stainless steel or titanium) or a polymer. Elongated member can be flexible or rigid (in portions other than flexible portion 60).

Motor 42 is preferably a stepper motor which rotates a predefined distance upon triggering of button 30.

Handle fastener mechanism 46 (shown in FIGS. 3b-c) includes a spur gear 70 meshed with spur gear 54. Gear 70 is rigidly attached to elongated member 58 and is driven by gear 54 in response to motor rotation. Elongated member 58 includes a connector 72 (e.g. hex-type connector) at its distal end. Connector 72 engages rod 73 (e.g. having an Allen interface) which is disposed within sleeve 75. Sleeve 75 is attached to flexible member 60 which is in turn connected to the distal portion of elongated member 58 via an Allen-hex interface 74.

FIGS. 4a-c illustrate shaft 14 in greater detail. Shaft 14 includes a coupling region 24 for engaging shaft 12 as well as drive tube 55 and elongate member 58 to handle 12.

Distal portion 18 is shown in greater detail in FIG. 4c, while coupling region 24 is shown in greater detail in FIGS. 4d and 5b.

FIGS. 4a, 4b and 4c shows distal portion 18 in its integrated configuration being rigidly attached to shaft 16. FIGS. 4d and 5b show handle attachment collar 300 and coupling element 301 thereof. When collar 300 is fully engaged and attached to coupling mechanism 38, shaft 65 and coupling element 301 are engaged and ready to transfer torque to distal portion 18 via shaft 65 and articulation activation via coupling element 301.

FIG. 5a illustrates articulation region 20 showing mechanism 84 for transferring rotation of drive tube 55 into articulation at hinge 86. FIG. 5a also illustrates flexible portion 60 of elongated member 58.

Flexible portion 60 of elongated member 58 is configured for compensating for changes in distances across the hinge region upon articulation of distal portion 18 with respect to proximal portion 16. In that respect, flexible portion 60 is fabricated as an elastic structure that can lengthen and shorten without losing rotational rigidity. For example, flexible portion 60 can be fabricated as a closely packed coil, a multi strand stainless steel or titanium cable or a tube having cutouts along its length which allow the tube to elastically bend.

Alternatively, compensation for changes in distances across the hinge region upon articulation of distal portion 18 can be effected using a sliding sleeve in proximal portion 16 of shaft 14.

FIG. 5d (which is also described above) illustrates a sliding-sleeve type shaft which includes a rod 73 which is disposed within sleeve 75 which is in turn attached to flexible member 60. Rod 73 can slide back and forth within sleeve(s) 75 to compensate for any changes in the angle of flexible portion 60. Thus rather than compensating for angulation by shortening or lengthening flexible portion 60, this embodiment of the present invention provides compensation within proximal portion 16 of shaft 14.

Mechanism 84 includes two perpendicularly-positioned gears a crown gear 88 and a spur gear 90. As is illustrated in FIG. 5a, flexible portion 60 of elongated member 58 runs through crown gear 88 (and is co-axial therewith) and parallel to spur gear 90.

FIG. 5c illustrates articulation region 20 with elongated member 58 and flexible portion 60 removed in order to more clearly show the arrangement of gears 88 and 90 of mechanism 84.

Crown gear 88 forms an end portion of drive tube 55 and is thus rotated with rotation of drive tube 55. Gear 88 perpendicularly engages gear 90 and as such rotation of gear 88 rotates gear 90 in a plane perpendicular to the longitudinal axis of shaft 14. Gear 90 engages gear 92 which is part of hinge region 86. Rotation of gear 92 (via gear 90) angulates distal portion 18 with respect to proximal portion 16 around hinge 86 and thus results in articulation of shaft 14. The gear ratio between the articulation gears can be 1:1.

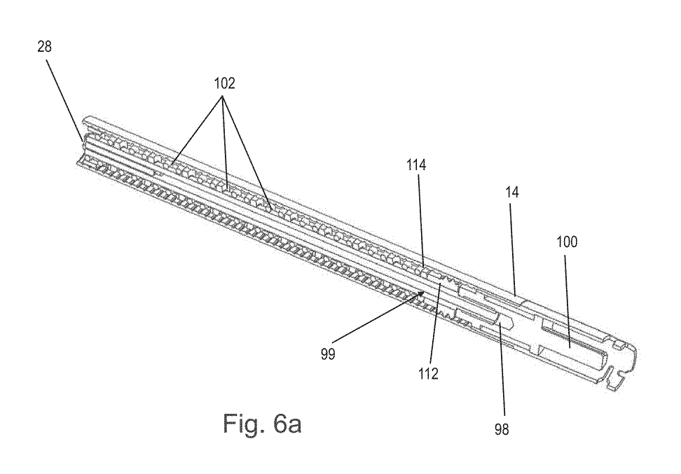

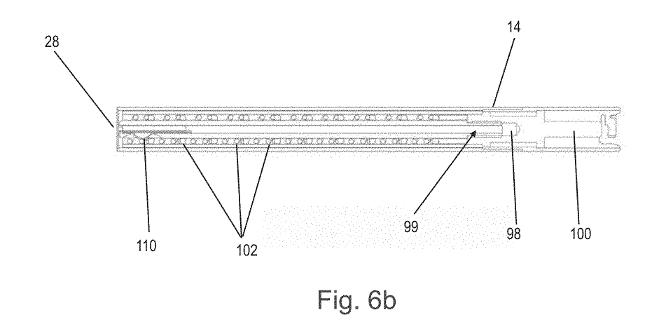

As is shown in FIG. 5c, articulation region 20 of shaft 14 also includes a coupling region 94 for distal portion 18 (not shown). Coupling region 94 serves two functions, coupling of distal portion 18 and included cartridge 20 to articulation region 20 of shaft 14 (thus connecting proximal portion 16 to distal portion 18) and coupling of elongated member 58 to a fastener drive mechanism 99 of cartridge 20 (FIGS. 6a-b). The latter can be achieved via mating of a hex socket 98 to an Allen pin 100 (of fastener drive mechanism).

Distal portion 18 and cartridge 20 are shown in greater detail in FIG. 6b. Ten fasteners 102 are shown loaded within cartridge 20. Pin 100 engages hex socket 98 of region 20 to enable rotation of fastener drive mechanism 99 via elongated member 58. Release of fasteners 102 is affected as follows.

Allen pin 100 is rigidly attached to elongated threaded member 114. A rotating nut 112 is threadably engaged to elongated threaded member 114. Rotating nut 112 includes a protrusion on either side for engaging longitudinal slotted openings in elongated threaded member 114. When Allen pin 100 rotates inside shaft 14, rotating nut 112 moves forward within the longitudinal slotted openings in elongated threaded member 114 causing the tacks in front of rotating nut 112 to move forward and be deployed into the tissue. Spring clip 110 prevents unintended expulsion of the tacks by applying minimal pressure on the most distal tack until the tack is deployed as described above.

Several types of fasteners 102 can be used along with device 10 of the present inventions. FIGS. 7a-d illustrate several examples of such fasteners which can be fabricated from a metal alloy (e.g. titanium, stainless steel) or a polymer (e.g. nylon). Fastener 102 can be fabricated from poly-lactic and/or -glycolic acid to enable biodegradation. Fasteners 102 include a tissue piercing end 104 (surgical needle type bevel) at a distal end of fastener body 106. Fastener body 106 is preferably shaped from a round or square wire forming a base measuring about 3.6 mm² and a coil measuring 4.0 to 6.0 mm in length. The tack can have a pitch of 1.2 to 1.8 mm.

As is mentioned hereinabove, device 10 of the present invention can be used in a variety of fully open or minimally invasive medical procedures.

One preferred use for device 10 is tacking of a mesh in minimally invasive repair of an inguinal hernia.

Following insertion of a mesh via a working port and positioning of the mesh against the abdominal wall the device of the present invention is turned on and the shaft of choice is selected and attached to the handle. A cartridge is then attached to the shaft via the bayonet quick connect fitting. After verifying the shaft is straight, it is then inserted into the abdominal cavity via a standard access port with the appropriate size opening. The mesh is deployed via a dedicated port and held in position via a grasper, the shaft is then articulated such that the cartridge distal end is pressed perpendicularly against the mesh and the abdominal wall. The tack firing button is then actuated and a single tack is deployed into the mesh and tissue. The firing button is then released and the cartridge is repositioned at the next tacking location to deliver the next tack. This process is repeated until the mesh is satisfactorily attached, the shaft is then straightened and removed from the body.

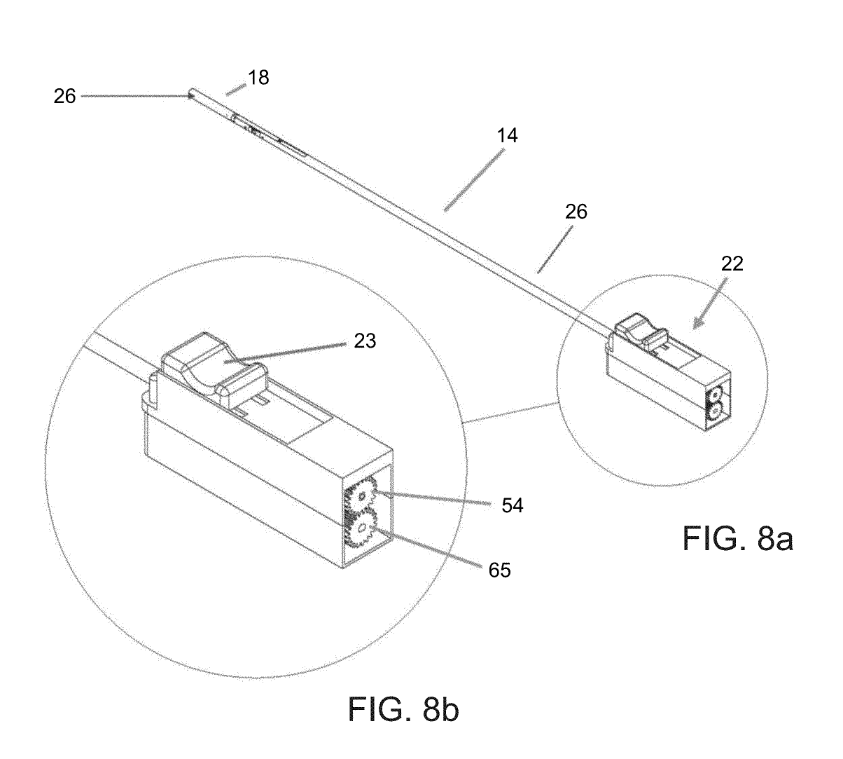

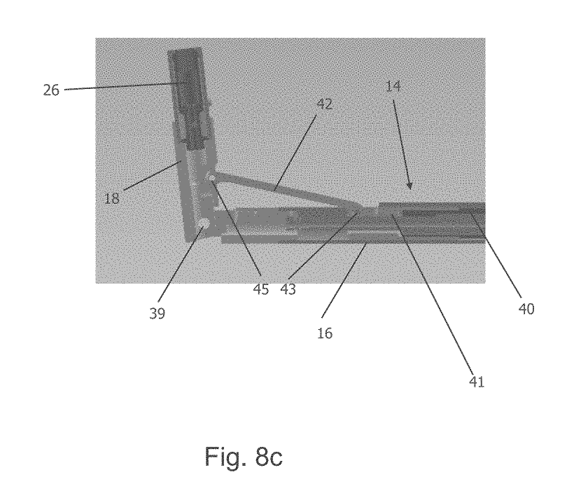

FIGS. 8a-c illustrate an alternative embodiment of a shaft articulation unit which includes shaft 14 (composed of proximal portion 16 and distal portion 18), cartridge 26, articulation control unit 22 and power transfer gears 54 and 65. Unit 21 is a self contained unit which can be disposable thus lowering the wear of the power transfer unit and simplifying the use of the device. Unit 22 of this embodiment is based on a slider mechanism which is controlled via a slider button 23. Sliding button 23 forwards (in the distal direction) and backwards (in the proximal direction) articulates the distal portion of shaft 18. Unit 21 can be connected to device 10 via a snap and lock interface, a twist and lock interface or any other mechanical coupling mechanism known in the art.

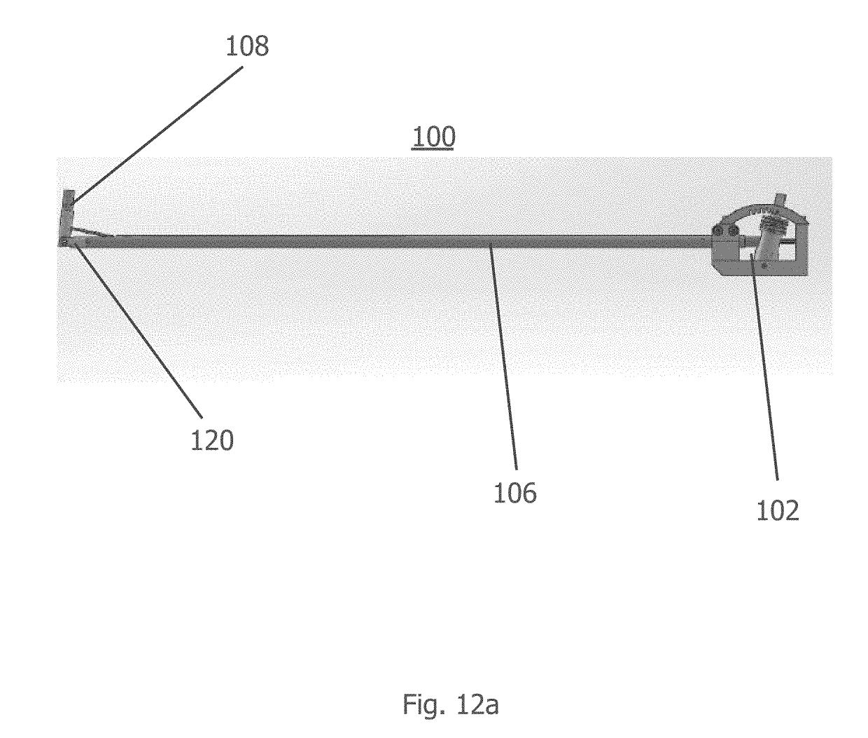

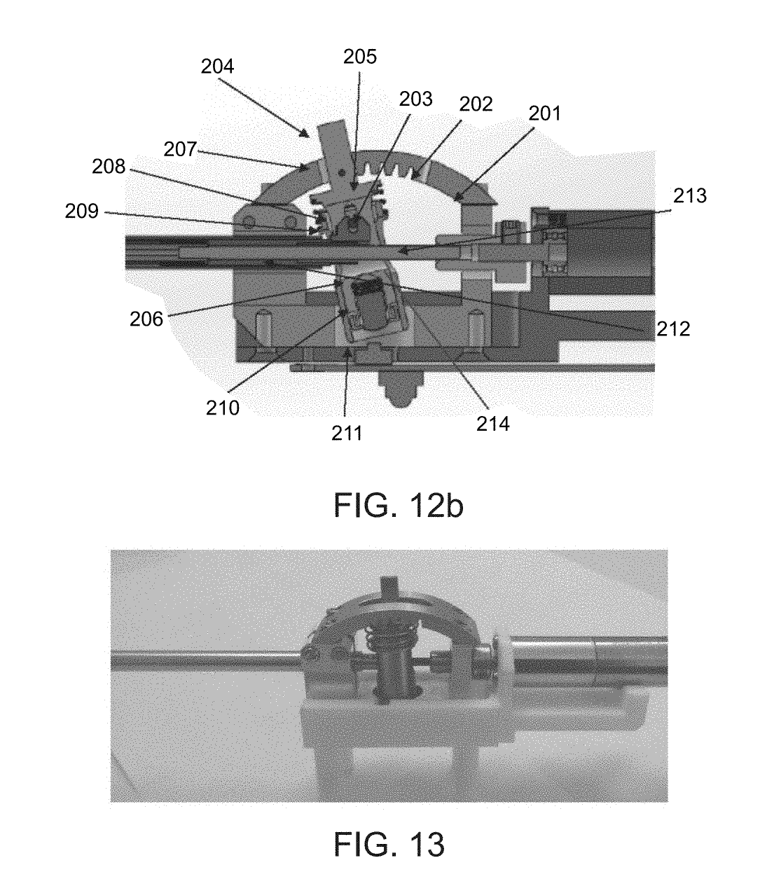

The articulation region of this configuration is shown in FIG. 8c. Proximal portion 16 and distal portion 18 (with cartridge 26) of shaft 14 are hingedly connected at 39. The proximal end of a push/pull rod 40 is connected to articulation control unit 22 (FIGS. 8a-b) or to articulation control mechanism 102 (FIGS. 12a-c). Rod 40 runs through a longitudinal lumen of proximal portion 16 and its distal end is connected to slider 41 which is in turn hingedly connected to strut 42 at hinge 43. The distal end of strut 42 is hingedly connected to distal portion 18 at hinge 45 which is distal (along shaft 14) to hinge 39. As such, when rod 40 is pulled towards the user (using the sliding button of articulation control unit 22 or by rotating assembly 214 described below) distal portion 18 pivots around hinge 39 and distal portion 18 angles with respect to proximal portion 16.

FIG. 12a-b illustrate yet another embodiment of a shaft articulation unit. In this embodiment, shaft articulation is controlled by a user through an interface provided on the proximal portion of the shaft.

FIG. 12a illustrates an articulated exchangeable shaft 100 (also referred to hereinunder as shaft 100) having a proximal portion 106 attached to a distal portion 108 through an articulation region 120. Articulation region 120 of shaft 100 can be any of the articulation regions described hereinabove (strut or gears). Shaft 100 also includes an articulation control mechanism (and interface) 102 located at a proximal portion 104 of shaft 100. Shaft 100 is attachable to a handle for providing functions such as tissue fastener delivery (the handle can be similar to handle 12 described hereinabove but without articulation control). Shaft 100 also can also include a micro switch which is activated when shaft 100 is coupled to a handle; the micro switch allows use of the handle with shaft 100 (similar to that described hereinabove for device 10).

FIG. 12b illustrates the internal components of articulating mechanism 102 of shaft 100.

Articulating mechanism 102 includes a frame 201 having slots 202 on an inner side of an upper bridge section. Mechanism 102 further includes an external articulation piston 203 (hereinafter piston 203) and an internal articulation piston 204 (hereinafter piston 204). Pistons 203 and 204 are actuatable against springs 205 and 206 (respectively).

Pushing piston 204 down (manually) against an upper spring 205 releases articulation lock pin 207 (hereinafter pin 207) from slot 202 in the upper bridge of frame 201.

Release of pin 207 enables manual rotation of assembly 214 around a pivot point (not shown) at the bottom of piston 203. Rotation (left to right in the view shown in FIG. 9b) of assembly 214 is transferred through an articulation movement transfer pin 208 to an articulation movement connector 209 and articulation bar 212 and to articulation region 120 of shaft 100. Once a user selects the desired deflection angle for distal portion 108, piston 204 can be released to allow pin 207 to engage a specific slot 202.

When piston 204 is pressed down, it pushes down on spring 206 which in turn pushes down on lower piston 210. Since spring 205 has a higher spring force constant than spring 206, once lower piston 210 is pressed, an articulation disable micro switch 211 is actuated (pushed) to disable the handle motor trigger before pin 207 is released from a groove 202 to allow articulation angle setting.

As used herein the term “about” refers to ±10%.

Additional objects, advantages, and novel features of the present invention will become apparent to one ordinarily skilled in the art upon examination of the following examples, which are not intended to be limiting.

### EXAMPLE

Reference is now made to the following example, which together with the above descriptions, illustrate the invention in a non limiting fashion.

### Example 1

### Device Prototype

A prototype of the present device was developed in order to test various device parameters. FIG. 9 illustrates the various components of the prototype device.

The prototype device was initially used to test parameters such as motor requirements (torque and force that would enable tack delivery), control (PC board selection), device integrity (e.g. of shaft-handle interface and shaft) safety features, and human interface. Once these parameters were optimized, the device was utilized to test function (articulation and delivery).

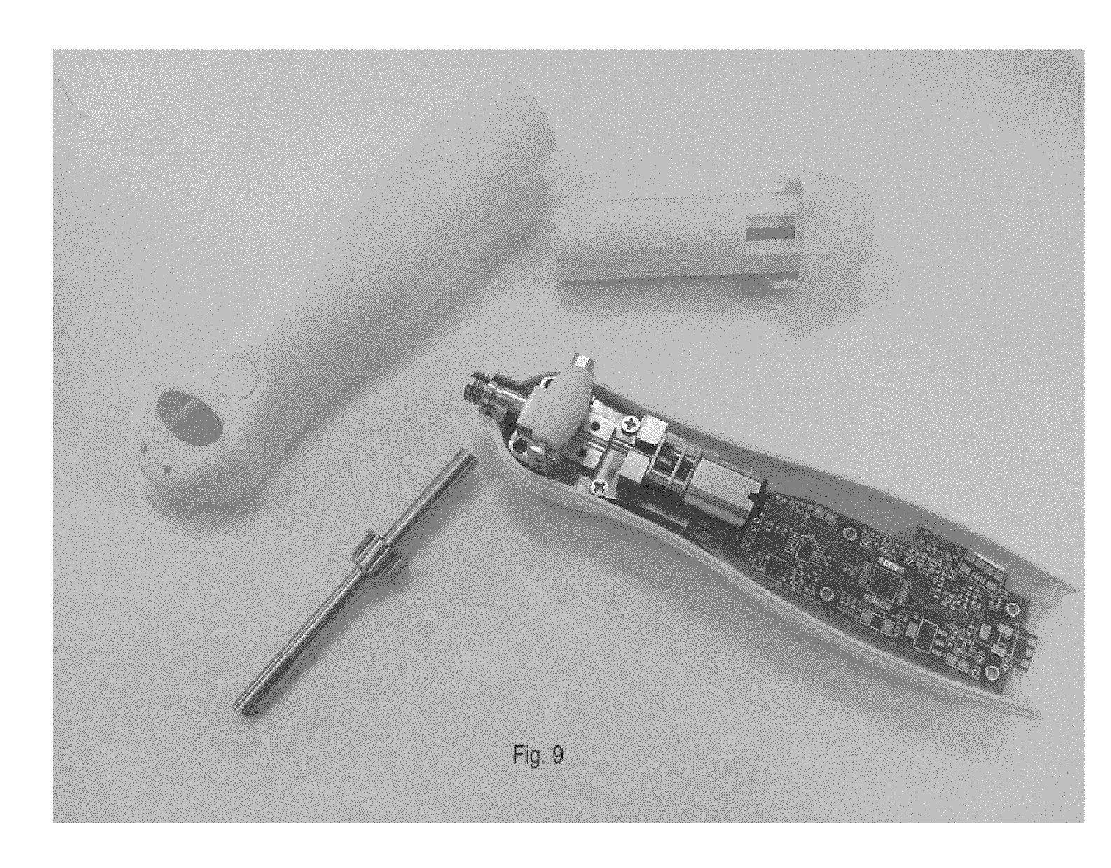

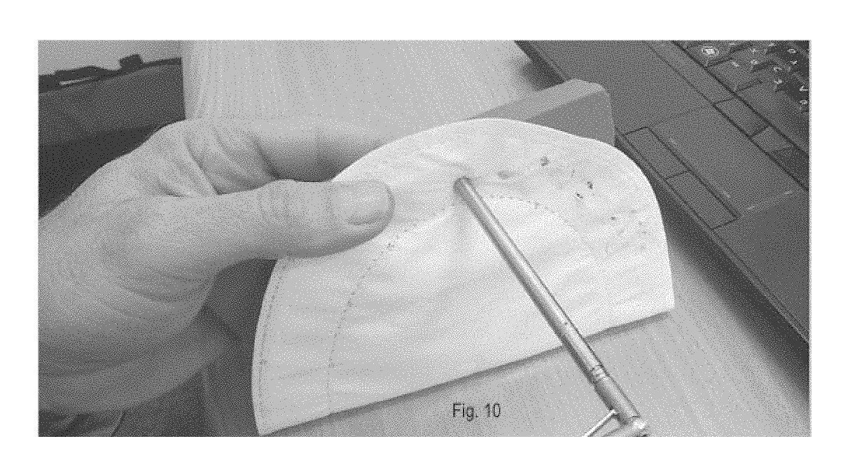

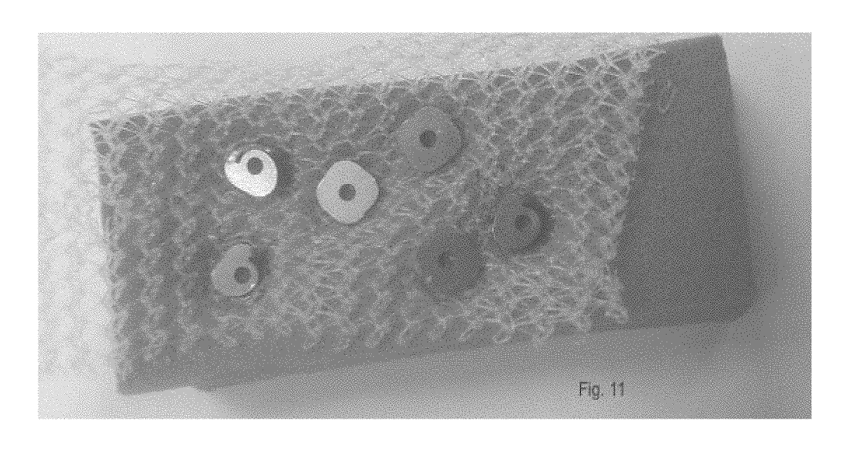

FIG. 10 illustrates tack delivery into a surgical mesh disposed over a material mimicking live human tissue. FIG. 11 illustrates the delivered tacks showing mesh fastening to the tissue-like material.

### Example 2

### Articulating Shaft Prototype

A prototype of an articulating shaft having a shaft-positioned articulation control mechanism and user interface (FIG. 13) was fabricated using standard CNC, Swiss type CNC and wire electro-erosion. A functional module was assembled and tested. Functional features, such as articulation control and torque delivery were successfully achieved.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-24. (canceled)

25. A medical device shaft attachable to a handle, the shaft comprising a proximal portion attached to a distal portion through an articulation region having an articulation control mechanism controllable from a proximal portion of said shaft, said articulation mechanism being for controlling an articulation angle of said distal portion of said shaft.

26. The medical device shaft of claim 25, wherein said articulation mechanism include a rod positioned in said proximal portion and being hingedly connected to said distal portion through a lever traversing said articulation region.

27. The medical device shaft of claim 25, wherein said articulation control mechanism is manually activatable to set an angle of articulation of said distal portion with respect to said proximal portion.

28. The medical device shaft of claim 26, wherein manually activating said articulation control mechanism actuates a switch for disabling functions of a handle attachable to said proximal portion of the shaft.

29. The medical device shaft of claim 26, further comprising a drive mechanism disposed within said shaft, said drive including an elongated member having a flexible region traversing said articulation region.

30. The medical device shaft of claim 29, wherein said flexible region of said elongated member traversing said articulation region is configured for accommodating a change in angle of said articulation region.

31. The medical device shaft of claim 29, wherein said flexible region is capable of elastically elongating when said distal portion is angled with respect to said proximal portion.

32. The medical device shaft of claim 29, wherein said flexible region forms an arc when said distal portion is co-linear with said proximal portion.

33. The medical device shaft of claim 29, wherein said drive mechanism further includes an implant driver disposed in said distal portion of said shaft.

34. The medical device shaft of claim 33, wherein a distal end of said elongated member engages said implant driver.

35. The medical device shaft of claim 34, wherein said implant driver is rotatable via said elongated member.

36. The medical device shaft of claim 35, wherein said distal portion of said shaft includes a plurality of implants.

37. The medical device shaft of claim 36, wherein rotation of said implant driver delivers and implant of said plurality of implants from said distal end of said distal portion.

38. The medical device shaft of claim 29, wherein said drive mechanism is only deployable when the shaft is attached to a handle.

39. The medical device shaft of claim 25, wherein said articulation control mechanism is a slide interface.

40. The medical device shaft of claim 39, wherein a position of said slide interface indicates an angle of said distal portion with respect to said proximal portion.

41. A medical device comprising the device shaft of claim 29 and a handle detachably connected to the shaft.

42. The medical device of claim 41, wherein said handle includes a motor for actuating said drive mechanism.
